(19) United States
(12) Patent Application Publication
(10) Pub. No.: US 2010/0022874 A1
(43) Pub. Date: Jan. 28, 2010

(54) Image Guided Navigation System and Method Thereof

(76) Inventors: Jaw-Lin Wang, Taipei City (TW); Yao-Hung Wang, Taipei City (TW); Been-Der Yang, Taipei City (TW); Chi-Lin Yang, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/507,855

(22) Filed: Jul. 23, 2009

(30) Foreign Application Priority Data
Jul. 25, 2008 (TW) 097128498

(51) Int. Cl. A61B 5/05; A61B 6/00

(52) U.S. Cl. 600/427

(57) ABSTRACT

An image guided navigation system comprises a memory, a locator, a processor and a display. The memory stores a plurality of CT images and a software program. The locator is capable of indicating a direction to a surgical area, and the indicated direction of the locator is defined as a first direction. The processor is electrically connected to the memory and the locator. At least one corresponding image corresponding to the first direction is obtained from the plurality of CT images by the processor executing the software program. The at least one corresponding image comprises at least one simulated fluoroscopic image. The display is capable of showing the at least one corresponding image.

### BACKGROUND OF THE INVENTION

1. Field of the Invention

The present invention relates to an image guided navigation system, and more particularly, to an image guided navigation system which uses a pointing direction of a locator to show a surgical image related to the pointing direction.

2. Description of the Related Art

Percutaneous spine surgery guided by fluoroscopy (dynamic X-ray image) is now common and causes less harm to the patient. The percutaneous puncture procedure is to apply a puncture needle having a diameter of 0.7 mm to pierce through a target site to reach a surgical area; then the puncture needle is used as a track for sending the medical device to the surgical area for treatment. Usually the diameter of the wound caused by the percutaneous spine surgery is less than 5 mm in diameter; therefore, the percutaneous spine surgery is categorized as a kind of the minimally invasive surgery. Although the percutaneous spine surgery can effectively reduce the operative trauma of the patient, it is a very dangerous and difficult technique since the surgeon cannot see the surgical area directly from the outside of the patient's body and must be careful when he/she uses a puncture needle to pierce through the patient's body.

The traditional puncture procedure is guided by X-ray images taken by a C-arm equipment and has two stages; the first stage is a direction control, and the second stage is a depth control. The direction control allows adjustment of a shooting angle of the C-arm equipment to make a projection of the spine anatomy on the fluoroscopic image with a special shape, such as the “Scottie dog,”, to help the surgeon determine the right direction; and the shooting angle is used as a puncture direction. Then the surgeon can pierce the puncture needle into the patient's body for a depth of 10 mm, and then subsequently proceeds with the depth control. The depth control allows adjustment of the C-arm equipment to take fluoroscopic images from a lateral side of the patient and to estimate the depth for the puncture needle to reach the surgical area; and then the puncture needle is guided to the surgical area.

Percutaneous spine surgery causes a smaller surgical incision than that caused by the traditional open surgery procedure and it uses planar X-ray images to determine the puncture direction, thus providing a more efficient method in clinical applications. However, if the surgeon does not have enough experience in performing the percutaneous spine surgery, he/she could have problem in determining a puncture site for treatment and need to repeat the procedure iteratively, which can prolong the surgery time and cause more wounds and higher radiation dosage on the patient. Moreover, the C-arm, would generate X-rays in taking X-ray images, and could expose the surgeon to excessive radiation, leading to health risk to the surgeon.

Therefore, in order to make the percutaneous puncture procedure a safe and efficient procedure, computer assisted navigation systems are developed to assist the procedure. Prior art techniques such as those disclosed in the US patent publications U.S. Pat. No. 6,165,181, U.S. Pat. No. 6,167,145 and U.S. Pat. No. 6,505,065 B1 have disclosed computer assisted navigation systems using pre-surgery CT images as guidance to help the surgeon perform the surgery in radiation-free environment. Furthermore, the CT images provide more accurate anatomical information than the overlapped fluoroscopic image for the surgeon to better identify the puncture site and to perform the surgery with higher precision. The computer assisted navigation system also has two control stages, namely the direction control and the depth control stages. The direction control is implemented by using an interface having four image windows on the display, which comprises a 3D spine image and three section images in fixed directions (the transverse section along the X-Y axes, the coronal section along the Y-Z axes, and the sagittal section along the Z-X axes). The 3D spine image can show the appearance of vertebrae and a virtual puncture needle to allow the surgeon to clearly see the moving puncture needle in relation to the surgical area on the spine and to ensure the puncture direction. However, the 3D spine image cannot show the internal structure of bones and other tissues such as blood vessels and nerves; therefore, the three section images must be provided to help the surgeon to correctly determine the best puncture path to avoid harming blood vessels and nerves in reaching the surgical area. When the direction is determined, the surgeon can pierce the puncture needle into the patient's body for a depth of 10 mm and then proceed with the depth control, which is implemented by monitoring the real-time location of the virtual puncture needle in the CT image to achieve precise positioning.

Although the computer assisted navigation system provides various advantages, a major drawback of the computer assisted navigation system is the complexity of direction control. As described above, the surgeon uses the direction control to handle four image data to construct a practical puncture site for the treatment. However, when the surgeon is under a great deal of stress and has to deal with multiple images at the same time, the process of the surgery could be hampered.

As to reducing the complexity of the direction control, many computer assisted navigation systems have been proposed, such as US patent publications U.S. Pat. No. 5,694,142, U.S. Pat. No. 6,038,467, and U.S. Pat. No. 7,203,277B2. Methods disclosed in these patents propose a device which can show the patient and the image before surgery under a same viewing angle for comparison. An LCD device disposed between the surgeon and the patient provides a way for better observation so that the surgeon can observe the CT images or the simulated fluoroscopic images of different depths inside the patient's body by adjusting the direction of the LCD. These methods can help the surgeon adjust the observation direction intuitively and determine the puncture direction and position efficiently. However, this kind of device covers the surgical area of the patient, reduces space for surgery, and leads to inconvenience in operating the medical device.

### SUMMARY OF THE INVENTION

It is an object of the present invention to provide an image guided navigation system which can adjust an indicated direction of a locator to show an image of a surgical area corresponding to the direction.

In order to achieve the above object, the present invention discloses an image guided navigation system, which comprises a memory, a locator, a processor, and a display. The memory stores a plurality of CT images and a software program. The locator is provided for indicating a direction to the surgical area, wherein the indicated direction of the locator is defined as a first direction. The processor is electrically connected to the memory and the locator, wherein at least one corresponding image corresponding to the first direction is obtained from the plurality of CT images by the processor executing the software program, the at least one corresponding image comprises at least one simulated fluoroscopic image; and the display is capable of showing the at least one corresponding image. With the design of the present invention, the surgeon can change the viewing angle of the surgical area by adjusting the indicated direction of the locator and determine the puncture direction of the puncture needle according to the at least one corresponding image to improve surgery efficiency. Besides, by using the simulated fluoroscopic images, it is possible for the surgeon to perform a surgery in an environment with no radiation concern.

The present invention discloses an image guided navigation method for applying the image guided navigation system, the image guided navigation method comprising the following steps: obtaining a plurality of CT images of a surgical area of a patient; indicating a direction to the surgical area by a locator, wherein the indicated direction of the locator is defined as a first direction; obtaining at least one corresponding image corresponding to the first direction by processing the plurality of CT images, wherein the at least one corresponding image comprises at least one simulated fluoroscopic image; and showing the at least one corresponding image.

### BRIEF DESCRIPTION OF THE DRAWINGS

FIG. 1 illustrates a view of an image guided navigation system in the present invention;

FIG. 2 illustrates a flow of an image guided navigation method in the present invention;

FIG. 3 illustrates an operation view of the image guided navigation method applied in the image guided navigation system;

FIG. 4 illustrates a view of a first embodiment of the image guided navigation system showing at least one corresponding image;

FIG. 5 illustrates a view of at least one MPR image of the image guided navigation system;

FIG. 6 illustrates a view of a second embodiment of the image guided navigation system showing at least one corresponding image; and

FIG. 7 illustrates a view of a third embodiment of the image guided navigation system showing at least one corresponding image.

### DETAILED DESCRIPTION OF THE PREFERRED EMBODIMENT

The advantages and innovative features of the invention will become more apparent from the following detailed description when taken in conjunction with the accompanying drawings.

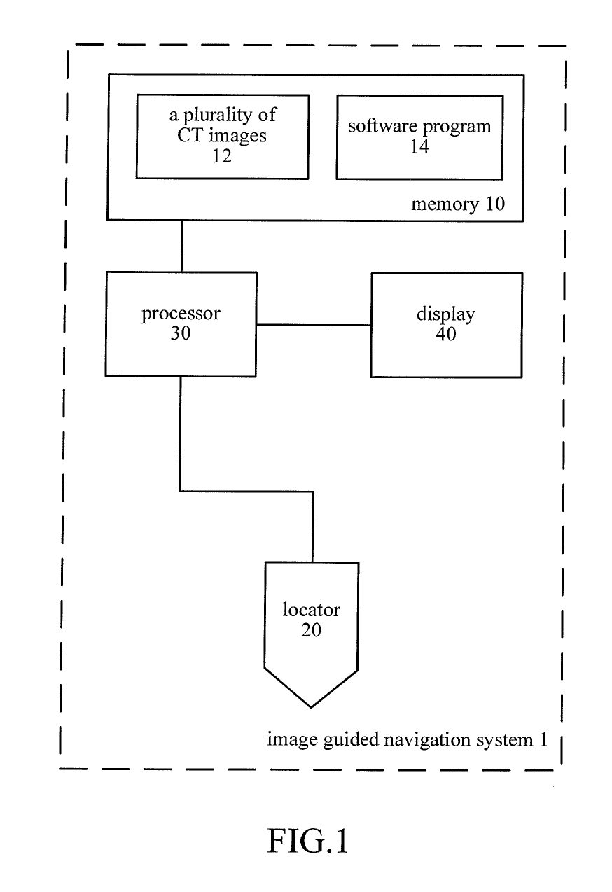

Please refer to FIG. 1 for a view of an image guided navigation system 1 in the present invention. The image guided navigation system 1 is applied in a surgery. A plurality of CT images of a surgical area of a patient is obtained before the surgery. As shown in FIG. 1, the image guided navigation system 1 comprises a memory 10, a locator 20, a processor 30 and a display 40. The memory 10 stores a plurality of CT images 12 and a software program 14. The locator 20 is provided for indicating a surgical area. The processor 30 is electrically connected with the memory 10, the locator 20 and the display 40 for instruction control and processing. The display 40 is provided for showing images. The locator 20 can be integrated with a puncture needle for the surgeon to perform the puncture procedure immediately after he/she confirms the puncture site to improve the efficiency; however, the present invention is not limited thereto.

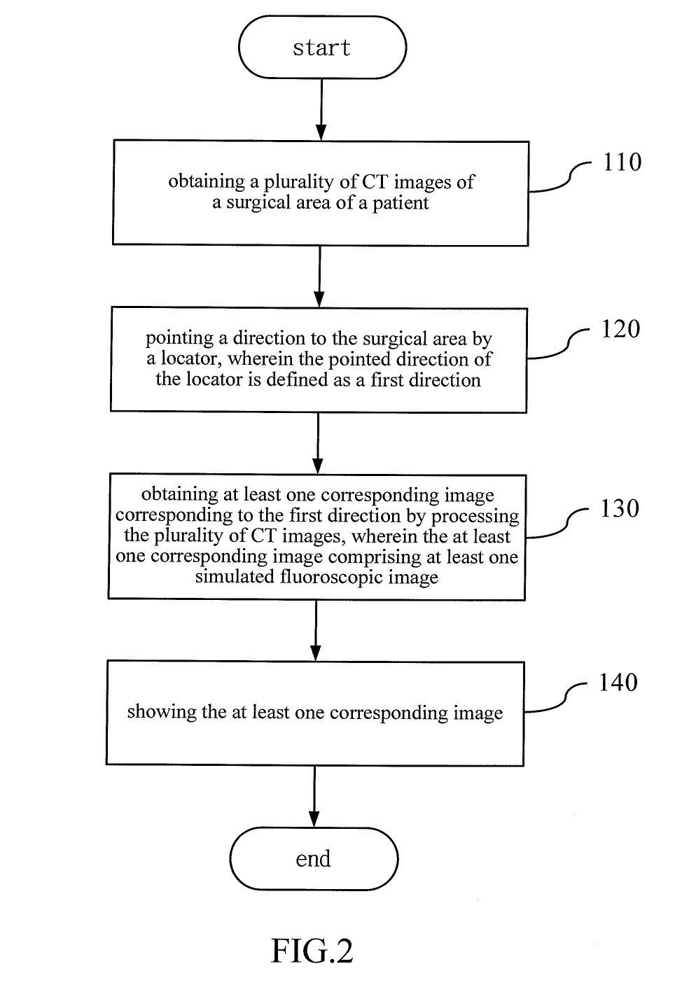

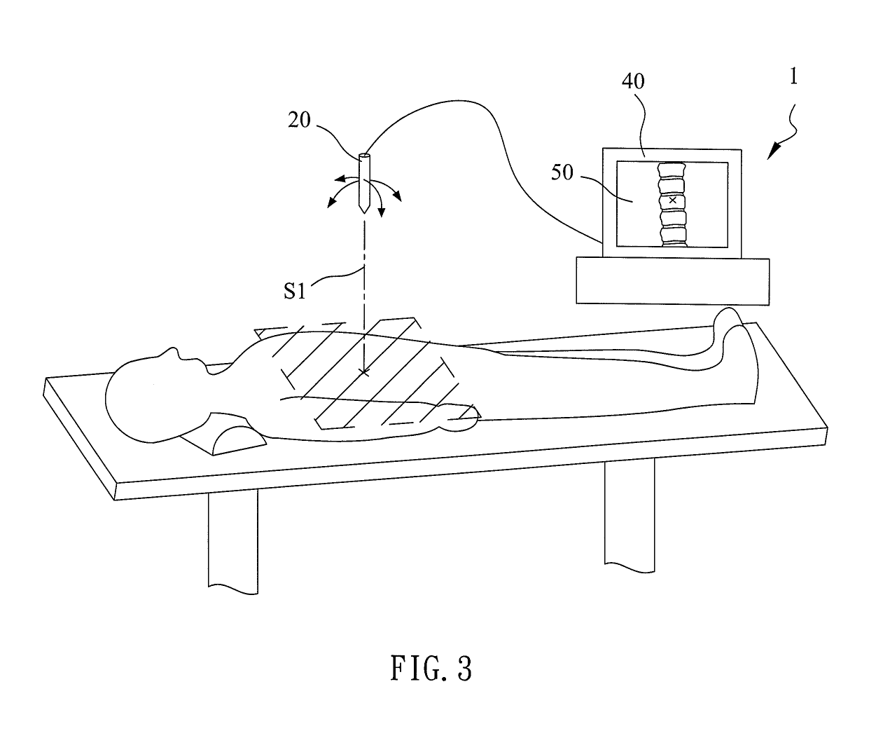

Please refer to FIG. 1 to FIG. 3. FIG. 2 illustrates a flow of an image guided navigation method in the present invention; FIG. 3 illustrates an operation view of the image guided navigation method applied in the image guided navigation system. As shown in FIG. 2, the image guided navigation method comprises steps 110 to steps 140, which will be described in detail as follows.
Step 110: Obtaining a plurality of CT images 12 of a surgical area of a patient.

As shown in FIG. 1, the image guided navigation method obtains the plurality of CT images 12 from the surgical area of the patient by using computed tomography before the surgery, and stores the plurality of CT images 12 in the memory 10 of the image guided navigation system 1.
Step 120: The locator 20 indicating a direction to a surgical area; wherein the indicated direction of the locator is defined as a first direction.

As shown in FIG.3, the image guided navigation system 1 comprises a locator 20; the locator 20 can point to any portion of the surgical area of the patient for positioning the puncture site of the surgery, wherein the indicated direction of the locator 20 is defined as a first direction S1. The locator 20 can be integrated with the puncture needle so as to carry out the puncture procedure in the surgery after the locator 20 has indicated the puncture site.
Step 130: obtaining at least one corresponding image 50 corresponding to the first direction S1 by processing the plurality of CT images 12, wherein the at least one corresponding image 50 comprises at least one simulated fluoroscopic image.

As shown in FIG. 1 and FIG.3, the locator 20 reports the information of the first direction S1 to the processor 30; the processor 30 can execute the software program 14 stored in the memory 10 to combine the plurality of CT images 12 to simulate a 3D configuration of the body tissues of the surgical area; then the processor 30 obtains at least one corresponding image 50 by using the 3D configuration of the body tissues of the surgical area corresponding to the first direction S1 indicated by the locator 20. The surgeon can use the at least one corresponding image 50 to understand the condition of the surgical area and the puncture site. The at least one simulated fluoroscopic image corresponding to the first direction S1 is obtained by using the software program 14 to generate X-ray photos of the surgical area with respect to the first direction S1.
Step 140: Showing the at least one corresponding image.

After the at least one corresponding image 50 corresponding to the first direction S1 is obtained, then the at least one corresponding image 50 is shown on the display 40. When there are more than one corresponding images 50, the plurality of corresponding images 50 can be simultaneously shown on the display 40 by executing the software program 14; the plurality of corresponding images 50 also can be selectively shown and switchable on the display 40, but the invention is not limited thereto. The at least one corresponding image 50 shown on the display 40 is changed by adjusting the first direction S1 to the surgical area indicated by the locator 20.

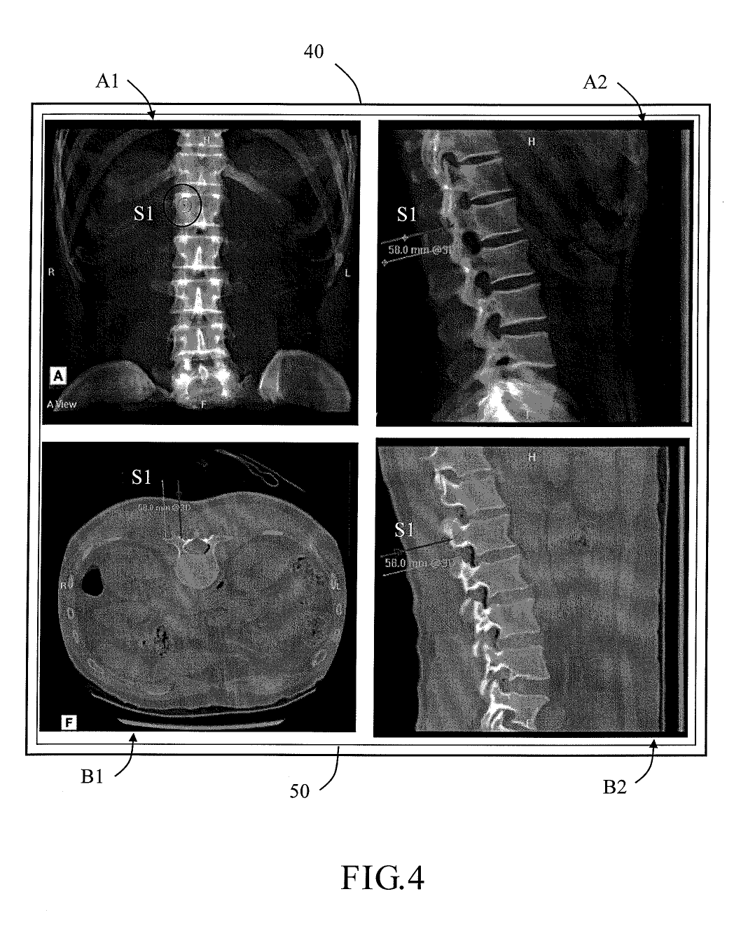

Please refer to FIG. 4 for a view of a first embodiment of the image guided navigation system showing at least one corresponding image. As described above, the image guided navigation system 1 can show the at least one corresponding image 50 on the display 40 by executing the software program 14. As shown in FIG. 4, the at least one corresponding image 50 comprises at least one simulated fluoroscopic image and at least one mutliplanar reconstruction (MPR) image. The at least one simulated fluoroscopic image uses the digital radiograph reconstruction (DRR) technique to simulate the superimposed X-ray image of a surgical area of a patient with respect to the first direction S1. In the first embodiment, the simulated fluoroscopic image comprises a viewing fluoroscopic image A1 and a lateral fluoroscopic image A2. The viewing fluoroscopic image A1 is obtained by using the first direction S1 as the shooting direction for the simulated fluoroscopic image technique; the viewing fluoroscopic image A1 is on a plane substantially vertical to the first direction S1. The present invention uses the first direction S1 as the viewing direction of the fluoroscopic image, which can simulate the images taken by the C-arm technique to let the surgeon obtain fluoroscopic images of different locations of the surgical area by using the locator 20. The lateral fluoroscopic image A2 is obtained by taking a simulated fluoroscopic image from a lateral side of the surgical area of the patient according to a designated position indicated by the first direction S1. The lateral fluoroscopic image A2 can simulate the lateral image of the patient taken by the traditional C-arm equipment and is provided for depth control. The simulated fluoroscopic image can prevent exposure of the surgeon from X-ray radiation caused by the traditional C-arm equipment and thus preserve the surgeon's safety.

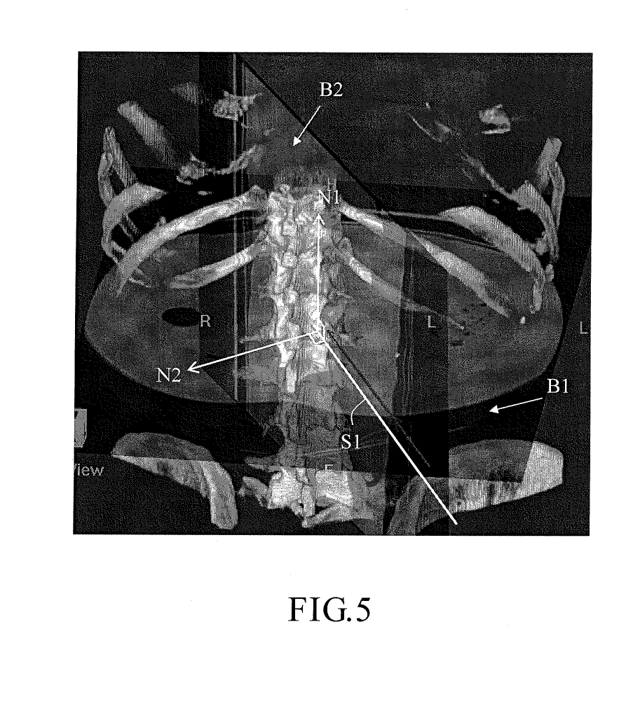

Please refer to FIG. 4 and FIG. 5. FIG. 5 illustrates a view of at least one MPR image of the image guided navigation system 1. As shown in FIG. 5, the image guided navigation system 1 obtains at least one corresponding image 50 corresponding to the first direction S1 from the plurality of CT images 14; the at least one corresponding image 50 further comprises at least one MPR image, which is on a plane along the first direction S1; furthermore, a normal line of the plane is substantially vertical to the first direction S1. The MPR technique can simulate 3D images of sections of the body tissues of the surgical area of the patient based on the first direction S1 and can obtain simulated section images with respect to the first direction S1. The at least one MPR image is constructed by a software with the plurality of CT images to help the surgeon identify the tissue sections clearly.

In this embodiment, the least one MPR image comprises a transverse section image B1 and a longitudinal section image B2; a normal line N1 of the transverse section image B1 and a normal line N2 of the longitudinal section image B2 is substantially vertical to the first direction S1. The transverse section image B1 simulates the transverse section of a front side of the surgical area of the patient; while the longitudinal section image B2 is substantially orthogonal to the transverse section image B1 to simulate the longitudinal section of a front side of the surgical area of the patient. Therefore, the image guided navigation system 1 can use the at least one MPR image with respect to the first direction S1 pointed by the locator 20 to help the locator 20 perform depth control; besides, the at least one MPR image can clearly show the section structures of body tissues to allow the surgeon to perform a puncture procedure without harming critical tissues. Furthermore, the at least one MPR image is substantially close to the axis of the human body to help the surgeon keep a sense of direction in performing the surgery.

As shown in FIG. 4, the at least one corresponding image 50 comprises the viewing fluoroscopic image A1, the lateral fluoroscopic image A2, the transverse section image B1, and the longitudinal section image B2 corresponding to the first direction S1; these corresponding images 50 are shown on four parts of the display 40 simultaneously. The viewing fluoroscopic image A1 can provide guidance for direction control in the puncture procedure; therefore, the surgeon can adjust the first direction S1 indicated by the locator 20 to determine a puncture site from the real-time viewing fluoroscopic image A1. Furthermore, the surgeon can use the lateral fluoroscopic image A2, the transverse section image B1, and the longitudinal section image B2 for depth control in the puncture procedure; he/she can clearly identify the tissue structures in the puncture path from the mutually orthogonal transverse section image B1 and the longitudinal section image B2 with respect to the first direction S1 indicated by the locator 20; he/she can also study the skeletal structures from a lateral side of the patient with the lateral fluoroscopic image A2 to control the puncture depth. Therefore, the precision and efficiency of the surgery are enhanced with the help of each corresponding image 50. It is noted that the arrangement and order of the corresponding images 50 on the display 40 matter only to the surgeon and can be adjusted according to the user's preference; the present invention is not limited to the embodiments disclosed in the present invention. Furthermore, the corresponding images 50 can be shown on the display 40 alone or in pairs and switched by hardware (such as a switching button) or the software program 14; however, the present invention is not limited thereto.

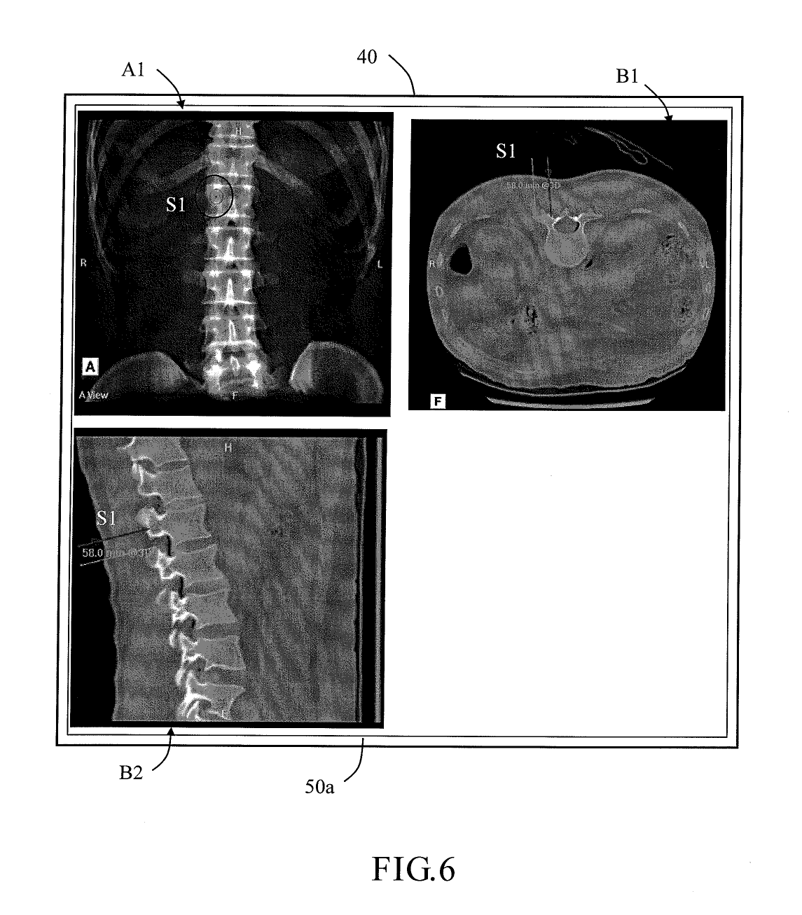

FIG. 6 illustrates a view of a second embodiment of the image guided navigation system 1 showing at least one corresponding image 50. This embodiment is a variation of the previous embodiment. As shown in FIG. 6, at least one corresponding image 50a comprises a viewing fluoroscopic image A1, a transverse section image B1, and the longitudinal section image B2. The viewing fluoroscopic image A1 is provided for determining a puncture direction, while the combination of the longitudinal section image B2 and the transverse section image B1 can completely show the tissue structures in the puncture path for determining the puncture depth; hence, the lateral fluoroscopic image A2 used for assisting depth control in the first embodiment is omitted, and the number of corresponding images 50a shown on the display 40 is reduced without affecting the precision in determining the puncture site.

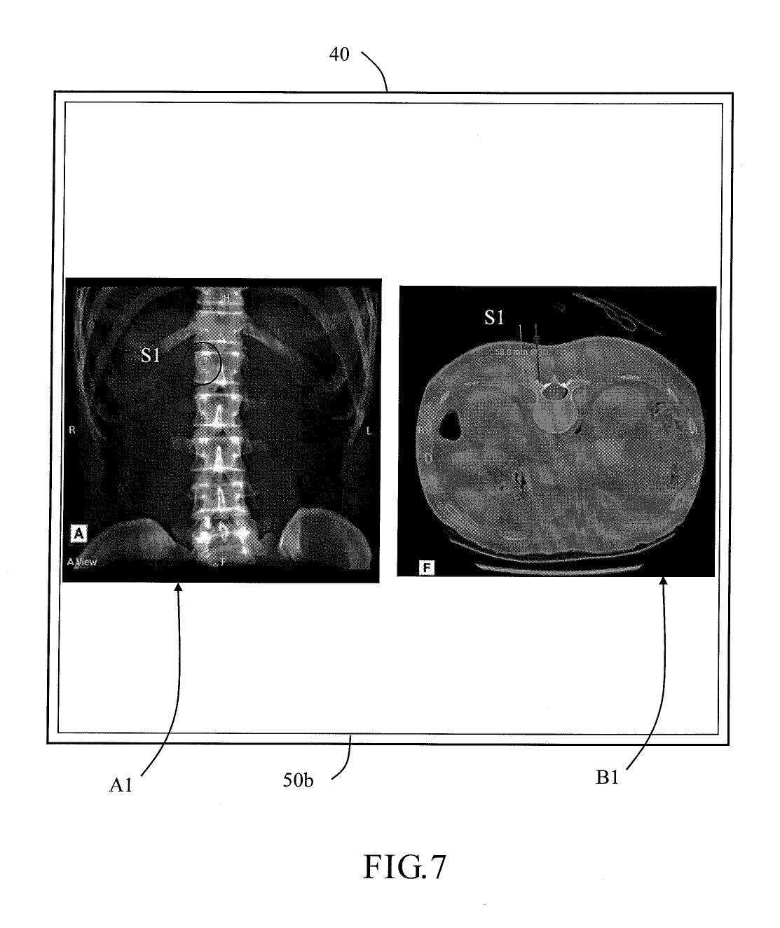

FIG. 7 illustrates a view of a third embodiment of the image guided navigation system 1 showing at least one corresponding image 50. This embodiment is a variation of the previous embodiment. As shown in FIG. 7, at least one corresponding image 50b comprises a viewing fluoroscopic image A1 and a transverse section image B1. In this embodiment, the viewing fluoroscopic image A1 is used for controlling the puncture direction; the transverse section image B1 is provided for depth control for the puncture procedure; and the longitudinal section image B2 used for assisting depth control in the second embodiment is omitted to further simplify the combination of the corresponding images 50b, but the necessary positioning function for the puncture procedure is still retained.

Furthermore, the corresponding images 50a, 50b in the second and third embodiment can be shown on the display 40 simultaneously by executing the software program 14; the corresponding images 50a, 50b also can be switched by hardware or by executing the software program 14, but the present invention is not limited thereto. It is noted that the arrangement and order of the corresponding images 50a, 50b on the display 40 matter only to the surgeon and can be adjusted according to the user's preference; the present invention is not limited to the embodiments disclosed in the present invention.

It is noted that the above-mentioned embodiments are only for illustration; it is intended that the present invention cover modifications and variations of this invention provided they fall within the scope of the following claims and their equivalents. Therefore, it will be apparent to those skilled in the art that various modifications and variations can be made to the structure of the present invention without departing from the scope or spirit of the invention.

What is claimed is:

1. An image guided navigation system for a surgery, wherein a plurality of CT images of a surgical area of a patient is obtained before the surgery, the image guided navigation system comprising:
a memory for storing the plurality of CT images and a software program;
a locator for indicating a direction to the surgical area, wherein the indicated direction of the locator is defined as a first direction;
a processor electrically connected to the memory and the locator, wherein at least one corresponding image corresponding to the first direction is obtained from the plurality of CT images by the processor executing the software program, the at least one corresponding image comprising at least one simulated fluoroscopic image; and
a display capable of showing the at least one corresponding image.

2. The image guided navigation system as claimed in claim 1, wherein the at least one corresponding image is changed by adjusting the first direction indicated by the locator to the surgical area.

3. The image guided navigation system as claimed in claim 1, wherein the at least one simulated fluoroscopic image comprises a viewing fluoroscopic image; the viewing fluoroscopic image is on a plane substantially vertical to the first direction.

4. The image guided navigation system as claimed in claim 3, wherein the at least one corresponding image further comprises at least one multiplanar reconstruction (MPR) image; the at least one MPR image is on a plane along the first direction, and a normal line of the plane is substantially vertical to the first direction.

5. The image guided navigation system as claimed in claim 4, wherein the at least one MPR image comprises a transverse section image.

6. The image guided navigation system as claimed in claim 5, wherein the at least one MPR image further comprises a longitudinal section image; the longitudinal section image is substantially orthogonal to the transverse section image.

7. The image guided navigation system as claimed in claim 6, wherein the at least one simulated fluoroscopic image further comprises a lateral fluoroscopic image; the lateral fluoroscopic image is obtained by taking a simulated X-ray photograph from a lateral side of the patient according to a designated position indicated by the first direction.

8. The image guided navigation system as claimed in claim 7, wherein the viewing fluoroscopic image, the lateral fluoroscopic image, the transverse section image, and the longitudinal section image are simultaneously and respectively shown on four different parts of the display.

9. The image guided navigation system as claimed in claim 7, wherein the viewing fluoroscopic image, the lateral fluoroscopic image, the transverse section image, and the longitudinal section image are switchable and selectively shown on the display.

10. The image guided navigation system as claimed in claim 1, wherein the display can simultaneously show a plurality of the at least one corresponding images.

11. The image guided navigation system as claimed in claim 1, wherein the display can switch between the plurality of the at least one corresponding images.

12. The image guided navigation system as claimed in claim 1, wherein the locator can be integrated with a puncture needle.

13. An image guided navigation method for a surgery, the image guided navigation method comprising the following steps:
obtaining a plurality of CT images of a surgical area of a patient;
indicating a direction to the surgical area by a locator, wherein the indicated direction of the locator is defined as a first direction;
obtaining at least one corresponding image corresponding to the first direction by processing the plurality of CT images, wherein the at least one corresponding image comprises at least one simulated fluoroscopic image; and
showing the at least one corresponding image.

14. The image guided navigation method as claimed in claim 13, wherein the at least one corresponding image is changed by adjusting the first direction pointed by the locator to the surgical area.

15. The image guided navigation method as claimed in claim 13, wherein the at least one simulated fluoroscopic image comprises a viewing fluoroscopic image; the viewing fluoroscopic image is on a plane substantially vertical to the first direction.

16. The image guided navigation method as claimed in claim 15, wherein the at least one corresponding image further comprises at least one mutliplanar reconstruction (MPR) image, the at least one MPR image is on a plane along the first direction.

17. The image guided navigation method as claimed in claim 16, wherein the at least one MPR image comprises a transverse section image.

18. The image guided navigation method as claimed in claim 17, wherein the at least one MPR image further comprises a longitudinal section image; the longitudinal section image is substantially orthogonal to the transverse section image.

19. The image guided navigation method as claimed in claim 18, wherein the at least one simulated fluoroscopic image further comprises a lateral fluoroscopic image; the lateral fluoroscopic image is obtained by taking a simulated fluoroscopic image from a lateral side of the surgical area of the patient according to a designated position indicated by the first direction.

20. The image guided navigation method as claimed in claim 19, wherein the viewing fluoroscopic image, the lateral fluoroscopic image, the transverse section image, and the longitudinal section image are simultaneously and respectively shown on four different parts of the display.

21. The image guided navigation method as claimed in claim 19, wherein the viewing fluoroscopic image, the lateral fluoroscopic image, the transverse section image, and the longitudinal section image are switchable and selectively shown on the display.

22. The image guided navigation method as claimed in claim 13, wherein the display can display a plurality of the at least one corresponding images simultaneously.

23. The image guided navigation method as claimed in claim 13, wherein the display can switch to show a plurality of the at least one corresponding images.

24. The image guided navigation method as claimed in claim 13, wherein the locator can be integrated with a puncture needle.
